(19) United States
(12) Patent Application Publication
(10) Pub. No.: US 2007/0106192 A1
(43) Pub. Date: May 10, 2007

(54) System and method for treating the spine with light therapy

(75) Inventor: Scot Johnson, Lutz, FL (US)

Correspondence Address: MCANDREWS HELD & MALLOY, LTD, 500 WEST MADISON STREET, SUITE 3400, CHICAGO, IL, 60661, US

(73) Assignee: Axiom Worldwide, Inc., Tampa, FL (US)

(21) Appl. No.: 11/233,756

(22) Filed: Sep. 23, 2005

(51) Int. Cl. A61F 5/00

(52) U.S. Cl. 602/32; 602/2

(57) ABSTRACT

A system for treating the spine of a patient by the application of tension to the spine. The system includes an alignment device secured to the patient, an actuator for producing tensile force, a patient interface device extending from the alignment device to the actuator and configured to apply the tensile force from the actuator to the spine of the patient through the alignment device, and a light therapy device positioned to irradiate a region of interest along the spine in conjunction with the application of the tensile force to the spine.

### BACKGROUND OF THE INVENTION

The present invention relates to a system and method for providing light therapy treatment to a patient's spine while the spine is placed under tension. More particularly, the present invention relates to a system and method for providing light therapy treatment to a patient's spine during traction or decompression therapy.

Therapists utilize spinal decompression therapy to treat various spinal ailments including herniated discs, degenerative disc disease, sciatica, posterior facet syndrome, and post surgical pain. Decompression therapy is a derivative of traction-based therapy, whereby the spine is placed into a state of tension by an outside force such as by a therapist manually or by an automated process. The spine is typically held in a continuous state of tension during traction-based therapy. Decompression therapy differs from traction therapy in that the traction applied to the spine is alternated typically between higher and lower sets of tension for predetermined periods of time. In either traditional traction or decompression therapy, spinal tension is typically maintained for periods of 30-minutes or longer.

As the spine is placed into a state of tension, the spinal vertebrae are separated in order to allow the intervertebral discs to realign into their proper positions. This action also allows herniated discs time to heal in a non-loaded state. During decompression therapy, interdiscal space is increased methodically for a period of time to promote healing. The increase in interdiscal space allows spinal discal segments to realign, rehydrate and return to a natural size, alleviates pinching of nerve bundles due to misaligned spinal vertebra, and provides a general unloaded state for which an environment for healing can continue. Additionally, nutrient-rich spinal fluid (nucleus pulposa) is drawn to the sites of tension via the pressure drop created by the separation of the vertebrae. This process involves further relaxing the paraspinal muscles, those muscles responsible for contracting the bony spinal vertebra, such that interdiscal space is increased correspondingly so. Para-spinal muscles react involuntarily to the ‘stretching’ of the spine by tensing in opposition to the force. Also, the conscious human patient may voluntarily and/or subconsciously flex the spinal muscles in reaction to tensile forces applied during the traction. Either or both patient reactions degrade the effectiveness of spinal therapy. Decompression therapy can overcome the reactions of the paraspinal muscles and subconscious flexing by cycling the tensile forces throughout the treatment period and thus confusing the paraspinal muscles.

Furthermore, therapists utilize light therapy to treat a number of ailments. Light therapy is the use of light sources such as, but not limited to, laser diodes and light emitting diodes (LEDs) to irradiate a region of the patient's body. The delivery of photon energy to the region is widely known to stimulate biological processes. Photo-biostimulation has been shown to be effective in the treatment of muscle and ligament injuries, inflammations, wounds, burns, chronic ulcerations including diabetic ulcers, deficient circulation, pain, nerve degeneration, eczema, shingles, infection, scars, acne, bone fractures, arthritis, osteo-arthritis, rheumatoidal arthritis, skin grafts, gingival irritation, oral ulcers, dental pain and swelling, cellulitis, stretch marks, skin tone, alopecia areata, trigeminal neuralgia, herpes, zosten, sciatica, cervical erosions, and other conditions.

While light therapy has been utilized to treat regions of a patient's body, conventional light therapy and irradiating devices are not typically used with treatments that articulate the regions to greater expose damaged tissues on a large scale. By incorporating a method for manipulating regions of interest of the patient's body to increase surface area of the regions exposed to light therapy, therapists are able to accelerate the healing process simply by increasing the photonic energy absorbed by the regions during a single treatment.

Therefore, a need exists for a spinal treatment system and method that includes light therapy to treat an injured spine.

### BRIEF SUMMARY OF THE INVENTION

Certain embodiments of the present invention include a system for treating the spine of a patient by the application of tension to the spine. The system includes an alignment device secured to the patient, an actuator for producing tensile force, a patient interface device extending from the alignment device to the actuator and configured to apply the tensile force from the actuator to the spine of the patient through the alignment device, and a light therapy device positioned to irradiate a region of interest along the spine in conjunction with the application of the tensile force to the spine.

Certain embodiments of the invention include a system for treating the spine of a patient by the application of tension to the spine. The system includes an alignment device secured to the patient, an actuator for producing tensile force, a patient interface device extending from the alignment device to the actuator and configured to apply the tensile force from the actuator to the spine of the patient through the alignment device, and a light therapy device. The alignment device is configured to retain the light therapy device such that the light therapy device is positioned proximate a region of interest along the spine and irradiates the region of interest.

Certain embodiments of the present invention include a method of treating the spine of a patient. The method includes positioning a light therapy device proximate a region of the body of the patient along the spine, applying tensile forces to the spine to increase separation between discs within the spine, and applying photonic energy from the light therapy device to the region of the body of the patient along the spine where the separation between the discs has been increased in order to increase photonic energy absorption at the region.

### BRIEF DESCRIPTION OF THE DRAWINGS

FIG. 1 illustrates a side view of a spinal treatment system according to an embodiment of the present invention.

FIG. 2 illustrates an isometric view of a light therapy device according to an embodiment of the present invention.

FIG. 3 illustrates a side view of a spinal treatment system used with the light therapy device of FIG. 2.

FIG. 4 illustrates a side view of a spinal treatment system according to an embodiment of the present invention.

FIG. 5 illustrates a side view of a head support system used for spinal treatment according to an embodiment of the present invention.

FIG. 6 illustrates a side view of the head support system of FIG. 5 used with a light therapy device according to an embodiment of the present invention.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentalities shown in the attached drawings.

### DETAILED DESCRIPTION OF THE INVENTION

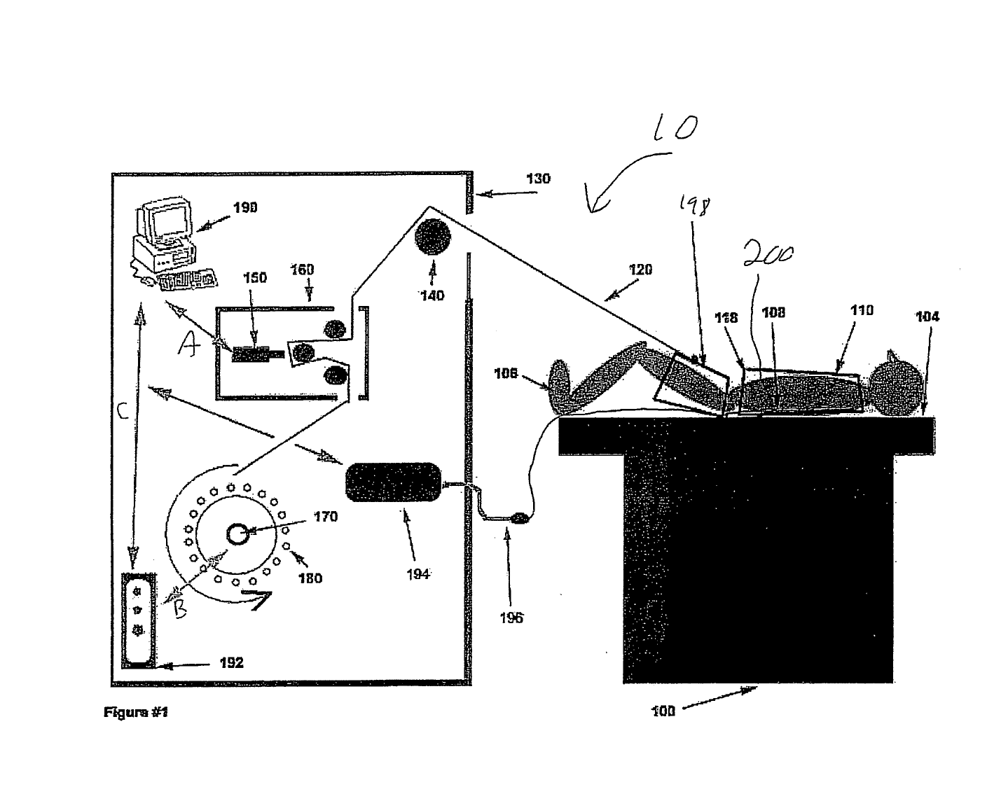

FIG. 1 illustrates a spinal treatment system 10 used to treat a patient 110 according to an embodiment of the present invention. By way of example only, the spinal treatment system 10 may be a traction or decompression treatment system. The system 10 includes a microprocessor, control system, or computing device 190 having firmware and/or software that operates to utilize and control an actuator 170. The computing device 190 is configured to interface with a user, such as by use of a monitor and keyboard setup. By way of example only, the actuator 170 may be electronically, hydraulically, pneumatically, or mechanically operated. The actuator 170 is connected to the patient 110 via a patient interface device 120 and harnesses 108 and 198. The system includes a separate controlling device 194 with an external connector 196 connected to a light therapy device 200. The controlling device 194 communicates with the main microprocessor or computing device 190 to control the operation of the light therapy device 200. This system 10 performs traction or decompression treatment by applying cycles of tensile forces from the actuator 170 on the spine 108 of the patient 110 through the interface device 120 in conjunction with the external light therapy device 200.

The patient 110 is positioned on a mechanical apparatus having a flat surface such as a bed or table 100. The bed 100 includes a head end 104 where the patient 110 lies his or her head and a base end 106 where the patient 110 lies his or her legs and feet. The bed 100 is positioned such that the patient 110 may be easily placed into alignment for treatment with the system 10. Additionally, the bed 100 may employ arm supports or rails to position the patient 110. The patient 110 wears an upper harness 108 that secures the upper body of the patient 110 to the bed 100. The patient 110 wears a lower body harness 198 that is connectable to the patient interface device 120. Alternatively, the patient 110 may wear any other appropriate securing device that is configured to connect the patient 110 to the interface device 120. The harness 198 may be connected to the patient interface device 120 through a clip or buckle that may alternately be secured and removed. The interface device 120 is configured to deliver and align tensile forces generated by the actuator 170 through the harness 198 along the spine 108 of the patient 110. The interface device 120 may be a strap, belt, or cable that is positioned relative to the patient 110 via a patient interface positioning device 140. The patient interface positioning device 140 may itself be moved to preferred positions by additional actuators.

The actuator 170 communicates with, and is controlled directly by, an actuator controller 192 as shown by arrow B. By way of example only the actuator controller 192 is a servo-amplifier 192. The actuator 170 may be attached to, or connected in line with, an encoder 180 that is capable of communicating motor shaft position and other motor metrics with the servo-amplifier 192. The servo-amplifier 192 may be capable of calculating any number of motor metrics, including work, position, distance, and rate and communicating those metrics to, and receiving them from, the computing device 190 as indicated by arrow C. The system 10 further includes a tensile force feedback system 160 which engages the interface device 120 between the actuator 170 and the harness 118. The tensile force feedback system 160 may include a load cell or dynamometer 150 that is positioned in line with the actuator 170 and is configured to provide feedback to the computing device 190 as indicated by arrow A.

The computing device 190 may be configured to communicate with the servo-amplifier 192, and the actuator 170, to monitor and correct as needed the resultant tensile force and motor metrics applied by the actuator 170 from the servo-amplifier 192. The computing device 190 may also be configured for use with a user interface system which communicates and deciphers the user's commands. This interface allows the user to structure treatment parameters. By way of example, all tension-producing and delivery apparatus are contained within a tower 130 located in such a position relative to the patient 110 so as to maximize tensile force delivery, and the tower 130 may include a user interface, such as a keyboard and monitor, for a technician to communicate with the system through the computer 190.

The light therapy device 200 may be located either in the bed 100 or in the lower harness 198 or upper harness 108. The light therapy controller 194 is connected to an external light therapy apparatus wire harness 196 that can be connected to the light therapy device 200. The light therapy controller 194 may include a microprocessor or computing components that communicates metrics and power to the light therapy device 200. Alternatively, the light therapy device 200 may contain a sufficient power source and controlling mechanism to operate independently of any external devices or may be operated wirelessly by a remote control. Upon activation, the light therapy device 200 irradiates the exposed portions of the patient's back during traction or decompression to provide bio-stimulation to the spine 108.

In operation, traction or decompression treatment begins by positioning the patient 110 correctly onto the bed 100. The patient 110 is outfitted with the lower harness 198 such that the patient 110 is connected to the patient interface device 120, and the harness 198 is configured to apply tensile forces generated by the actuator 170 to the spine 108 of the patient 110. The patient 100 is outfitted with the upper harness 118 to secure the upper body of the patient 110 to the bed 100. The configuration of the harnesses 118 and 198 allows the lower part of the patient's body to extend in line with the patient interface device 120 while the patient's upper body is anchored to the bed 100. The operator of the traction or decompression system 10 may use the patient interface system to select the proper treatment parameters for the traction or decompression therapy. Furthermore, the operator may communicate with the computing device 190 utilizing software and/or firmware. The computing device 190 and controller 192 then activate the actuator 170, which applies tensile forces to the interface device 120 and thus to the patient's spine through the harness 198.

The operator may also use the patient interface system to select a light therapy program for the patient during traction or decompression therapy. The light therapy program is communicated to the computing device 190 which in turn communicates the commands or program to the light therapy controller 194. The light therapy device 200 is then activated and irradiates the region of the patient's spine that is being treated with tensile forces.

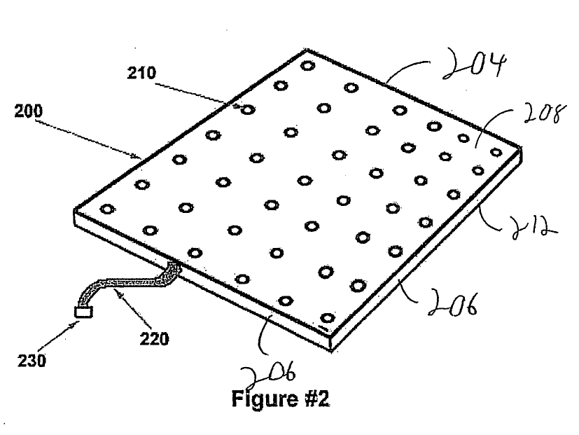

FIG. 2 illustrates an isometric view of the light therapy device 200 of FIG. 1. The light therapy device 200 is a flexible, conforming plate 204 having side walls 206 and top and bottom surface 208 and 212. The plate 204 includes an array of light sources 210 along the top surface 208. The supporting plate 204 may be made of flexible or inflexible materials, and/or conforming and stretchable materials. The plate 204 may additionally be water-resistant and may be configured to react to the patient's body heat. By way of example only, the plate 204 may be made of materials such as neoprene rubber or a gel-pad, which can provide water-resistant or waterproof properties while providing shape altering properties. The plate 204 is sufficiently thick to house the light sources 210 and may be hollow such that the light sources 210 are electrically and structurally interconnected within the plate 204.

The light sources 210 are configured to provide light and may be, by way of example only, laser diodes or light emitting diodes. The light sources 210 may be linked within the plate 204 by wires or by a flex circuit, for example. Alternatively, the light sources 200 may be contained in a single header, or multiple distributed headers, and the output of the light sources 200 may be routed through the top surface 208 of the plate 204 via conduits such as fiber optics or light pipes such that light is distributed to desired areas along the patient's body. The light sources 210 may be connected via an electrical network in series, such that all light sources 210 may be activated or deactivated simultaneously. Alternatively, the light sources 210 may be networked into groups or clusters, or activated individually, depending upon the desired irradiation pattern. A distributed system of light sources 210 along the top surface 208 may be beneficial by applying greater optical energies in one area than in another area in order to accommodate thermal differences across the light therapy device 200 due to heat generated by the light sources 210 or by the patient's body.

The light therapy device 200 includes a wire harness 220 with a connector 230 extending from the plate 204. The connector 230 is connectable to a power source/controller in order to provide power and data to the light therapy device 200 via the wire harness 220. For example, the harness 220 may communicate data such as intensity, frequency, on/off time, thermal data and pressure to the light therapy device 200 from a controller. By way of example only, the harness 220 may be connected to the external connector 196 of the controlling device 194 of FIG. 1. Alternatively, the light therapy device 200 may contain a power source and sufficient controlling mechanisms such that the light therapy device 200 can operate independent of an external controlling device or can be operated by wireless control. The wire harness connector 230 may be a flex circuit, a bundle of electrical wires, or may be a bundle of fiber optic connections. Where the wire harness 220 includes fiber optics, the connector 230 may itself contain light source(s) that are situated adjacent to the fiber optic wire harness 220 and inject photonic energy for delivery to the plate 204, which in turn directs the irradiating energy into regions of interest along the spine of the patient. One benefit of utilizing light sources 210 within the connector 230 and piping the light via fiber optics to regions of interest along the spine is the avoidance of excessive local heating that comes with positioning light sources 210 directly at or near the region receiving light therapy.

In operation, the light therapy device 200 is positioned under the spine 108 of the patient 110 for use with the traction or decompression treatment system 10 of FIG. 1. For example, as shown in FIG. 1, the light therapy device 200 is positioned beneath the lumbar spine to irradiate the lumbar spine during traction or spinal decompression treatment. The bottom surface 212 of the light therapy device 200 is positioned on the bed 100 and the top surface 208 is proximate the spine of the patient 110.

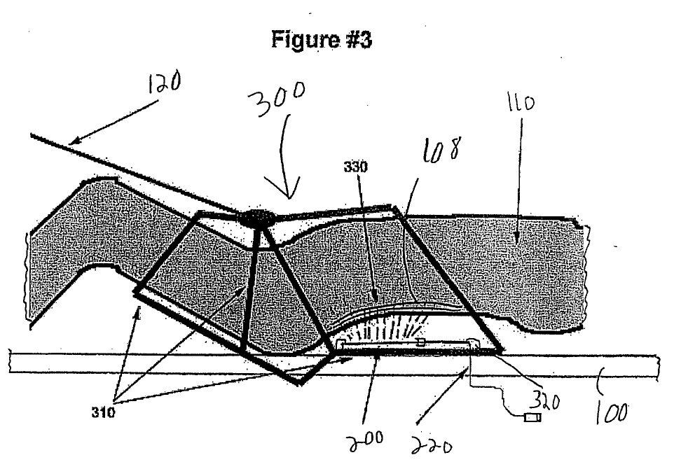

FIG. 3 illustrates a side view of a spinal treatment system 300 used with the light therapy device 200 of FIG. 2. The patient 110 is positioned on the bed 100 and within a lower body harness 310 that has been adapted to deliver traction or spinal decompression forces for treatment of the lumbar spine 108. By way of example only, the harness 310 is a textile harness. The lower body harness 310 is secured to the lower body of the patient 110 and is connected to the patient interface device 120. By way of example, the patient interface device 120 is a strap. The patient interface device 120 is in turn connected to a traction or decompression treatment system such as the system 10 shown in FIG. 1. The lower body harness 310 applies the forces delivered by the patient interface device 120 to the lower body while the upper body of the patient 110 is secured to the bed 100 and is rendered immobile during the traction or decompression treatment.

The lower body harness 310 includes a compartment 320 that is specifically designed to receive and retain the light therapy device 200 of FIG. 2 in such a position as to allow irradiation of the extended lumbar spinal segments 108 during traction or spinal decompression treatment. The wire harness connector 220 of the light therapy device 200 extends outside of the textile harness 310 for connection to a light therapy controlling system. Alternatively, the light therapy device 200 may be independently powered and/or wirelessly operated. The light therapy device 200 may be shaped and/or configured to conform to the contours of the patient's lumbar spine 108. The textile material of the lower body harness 310 allows the lower body and light therapy device 320 to flex within the harness 310 and compartment 320, respectively. The compartment 320 of the lower body harness 310 may have an opening located below where the lumbar spine 108 is positioned such that the light therapy device 200 directly contacts the skin of the patient 110 at the lumbar spine 208. Alternatively, the compartment 320 may include a clear barrier having sufficient spectrometric transmission qualities to allow for irradiating light 330 of the light therapy device 200 to pass through to the skin of the patient 110 along the lumbar spinal segments 108. The textile harness 310 and accompanying light therapy device 200 flex and contract with the forces of the traction or decompression treatment system such that the light therapy device 200 remains positioned under the lumbar spine 108 to irradiate the lumbar spine 108 with photonic energy 330 during the treatment.

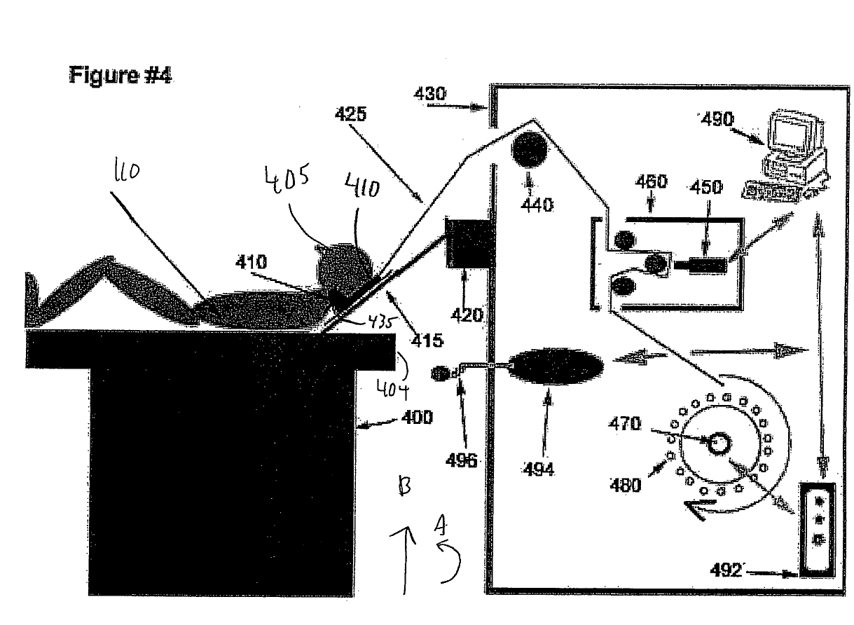

FIG. 4 illustrates a side view of a spinal treatment system 400. By way of example only, the system 400 may be a traction or decompression treatment system. The therapy system 400 includes many of the same components and operates similarly to the system 10 of FIG. 1. The system 400 includes a microprocessor or computing device 490 that utilizes and controls an actuator 470. The computing device 190 is configured to interface with a user, such as by use of a monitor and keyboard setup. The actuator 470 may communicate and be controlled directly by an actuator controller 492 that communicates with the computing device 190. The actuator 470 may be attached to or in line with an encoder 480 that is capable of communicating motor shaft position and other motor metrics with the controller 492. The controller 492 may be capable of calculating and/or communicating any number of motor metrics including work, position, distance, and rate to the computing device 490. Additionally, tensile force feedback systems 460 including a load cell or dynamometer 450 may be located within the region and in line with the actuator 470 for providing feedback to the computing device 490. The computing device 490 communicates with the controller 492 and/or the actuator 470 and monitors and corrects the resultant tensile force and motor metrics of treatment.

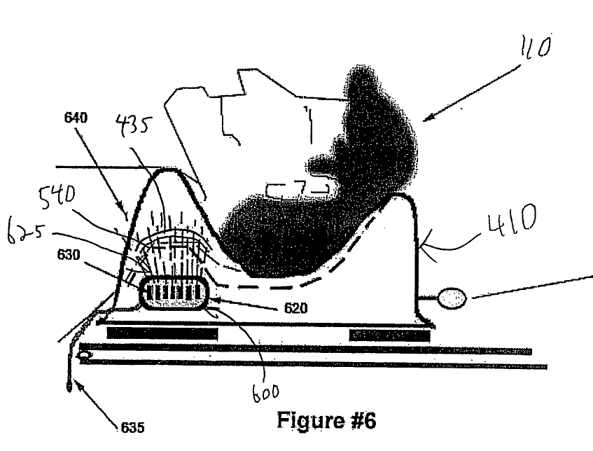

The system 400 also includes a separate controlling device 494 with an external connection 496 for a light therapy device 600 (FIG. 6). The controlling device 494 communicates with the main computing device 490 such that the operator can control light therapy through the computing device 490. The system 400 is substantially enclosed in a tower 430. The system 400 is used to perform traction or decompression treatment of the cervical spine 435 of the patient 110 in conjunction with the external light therapy device 600 embedded within a patient head adapter 410.

The system 400 includes a bed or table 100 that has a patient head adapter 410 positioned thereon at the head end 404 of the bed 100 to receive the head 405 of the patient 110. Additionally, the bed 100 may employ arm supports or rails to position the patient 110. The head adapter 410 is configured to provide a preferable distribution of forces to the patient's head 405, for example along the occipital lobes of the head 405. The patient 110 is connected to the electromechanical actuator 470 via a patient interface device 425 extending from the head adapter 410. The patient interface device 425 and head adapter 410 deliver and align the tensile forces generated by the electromechanical actuator 470 to the cervical spine 435 of the patient 110. The interface devices 425 may include a strap or cable that is positioned relative to the patient 110 via a patient interface positioning device 440. The positioning device 440 may itself be moved to preferred positions by additional electromechanical actuators. The head 405 of the patient 110 is kept in line with the tensile forces delivered by the interface device 425 by the patient head adapter 410. The patient head adapter 410 is configured to hinge or rotate relative to the bed 100 and in line with the patient interface device 425. The head adapter 410 is a conforming head support that is slidably connected to a head support platform 415. The platform 415 hinges about a stationary base of the bed 100 proximate the head end 404 of the bed 100 and at the region of the base of the neck. By way of example only, the platform 415 hinges proximate the C7/T1 cervical spinal location of the cervical spine 435. The head support platform 415 may be connected to the tower 430 via a connection point or block 420 that is raised and lowered in conjunction with the patient positioning device 440. As the connection block 420 is raised or lowered, the platform 415 rotates with respect to the bed 100 and cervical spine 435 accordingly.

In operation traction or decompression treatment on the cervical spine 435 of the patient 110 begins by positioning the patient 110 correctly onto the bed or table 100 with the patient's 405 head in the head adapter 410. The body weight of the patient 110 is sufficient to anchor the body of the patient 110 while the patient's head 405 is allowed to extend within the head adapter 410. The head adapter 410 is configured to receive and retain a light therapy device underneath the patient's neck at the cervical spine 435 such that the device delivers therapeutic irradiation to the neck along the extended cervical spinal segments 435. The operator of the traction or decompression system 400 may use the patient interface system to select the proper treatment parameters for the traction or decompression treatment. The operator may also enter data or treatment profiles for light therapy through the patient interface system to the computing device 490 in order to activate and control the light therapy controller 494.

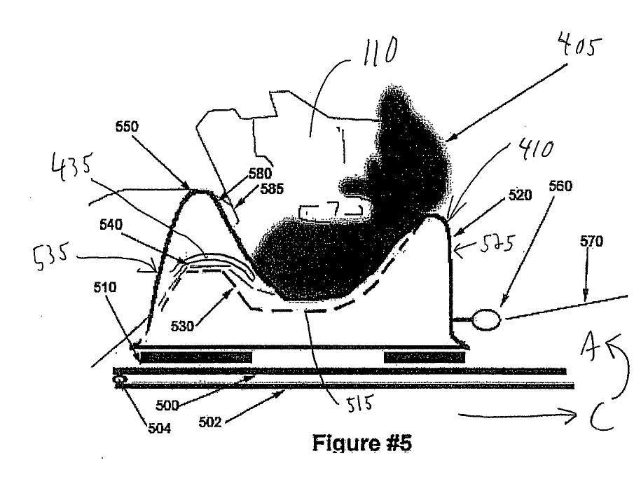

FIG. 5 illustrates a side view of the head adapter 410 used with the traction or decompression system 400 of FIG. 4. The head adapter 410 is used to secure the head 405 of the patient 110 during treatment and comfortably and effectively deliver traction or decompression forces to the cervical spine 435 of the patient 110. This head adapter 410 includes a conformable head support 520 at a top end 525 of the head adapter 410 that sufficiently positions the patient's neck 540 for traction or decompression treatment with the system 400 of FIG. 4. The head support 520 may be made of a material such as injection molded urethane foam, rubber foam, foam with gel integrals, plastic of a comfortable nature, or any material that substantially cradles the patient's head 405 and neck 540 in a comfortable manner. Patient comfort with regards to cervical traction or decompression treatment is essential because of the added obstacle of para-spinal muscle contraction due to the psychological claustrophobia caused by the head adapter 410. The head support 520 includes a bowl 515 and arch 530 that receive the patient's head 405 and follow the shape of the patient's head 405 and the natural line of the neck 540. Because the morphology of human head and neck structures differs among patients, it may be preferable that the conforming head support 520 be detachable from the head adapter 410 and that various sizes of conforming head supports 520 be interchangeable for use on the head adapter 410.

The head adapter 410 also includes occipital horns 550 on opposite ends of a bottom end 535 of the head adapter 410. The occipital horns 550 define a channel therebetween that receives the patient's neck 540 and are positioned to engage the occipital lobes 585 of the patient 110 at occiput contact surfaces 580. The horns 550 may be made of any number of conformable, flexible materials such that the patient's occipital lobes 585 are comfortably engaged by the horns 550. Thus, the patient's head 405 is secured within the head adapter 410 between the head support 520 and the occipital horns 550.

The conformable head adapter 410 is secured to a head support platform 500 that rides on table slides 510. By way of example only, the table slides 510 may be rectangular planes supported by thin ball bearing rails. The head adapter 410 is configured to slide in a direction longitudinally aligned with the patient's neck 540 along the platform 500. The head adapter 410 is configured to travel a sufficient distance to adequately extend the cervical spine 435 for traction or decompression treatment. The table slides 510 are able to travel several inches in the direction of tensile force applied to the head adapter 410, and the table slides 510 secure the head adapter 410 to the head support platform 500. The conformable head support 520 and table slide elements 504 also contain an eyehook 560 that is configured to be attached to a patient interface device 570 which is connected to a traction or decompression treatment system such as the system shown in FIG. 4 that delivers tensile forces to the cervical spine 435.

The head support platform 500 is connected to a stationary base 502 by a hinge 504 at a region of the base 502 proximate the C7/T1 cervical spinal location of the patient's neck 540. By rotating the platform 500 about the hinge 504 in the direction of arrow A, the conformable head adapter 410 and table slide elements 504 likewise are rotated upwards in the direction of arrow A, flexing the cervical spine 435 to a preferred alignment for traction or decompression treatment and improving patient comfort. The head adapter 410 positions the neck 540 into a convex position relative to the stationary base 502. Alternatively, the head adapter 410 may be attached to the bed 100 of FIG. 4 instead of the stationary base 502 or the stationary base 502 may be connected to the bed 100, such that the rotated head adapter 410 positions the neck 540 of the patient 110 into a convex position relative to the bed 100. Furthermore, the patient head adapter 410 may be attached to the bed 100 and to the connection block 420 (FIG. 4) of the tower 420 (FIG. 4) located behind the patient's head 405. Referring to FIG. 4, the connection block 420 may be operated to move upward in the direction of arrow B to rotate the head adapter 410 in the direction of arrow A and thus lift the patient's head 405 relative to the hinge 504 (FIG. 5) located near the base (C7/T1 spinal vertebral location) of the patient's neck 540 (FIG. 5) such that the patient's neck 540 is in a convex position relative to the head support 520 (FIG. 5). A light therapy device may be positioned underneath this convex region of the patient's neck 540 to provide light therapy to the cervical region 435 of the spine during traction or decompression treatment.

Returning to FIG. 5, in operation, once the patient's head 405 is secured in the head adapter 410 and, if necessary, the head adapter 410 is rotated about the hinge 504 to a desired location, the traction or decompression system 400 (FIG. 4) is activated. The patient interface device 570 applies tensile forces to the head adapter 410, pulling the head adapter 410, and the patient's head 405, in the direction of arrow C to decompress the cervical spine 435. As in various massage and chiropractic modalities, tensile forces are applied to the patient's head 405 and neck 540 through the occipital lobes and occiput bony structures 585 of the human head, which are engaged by the occipital horns 550 as the head adapter 410 is pulled in the direction of arrow C. The occipital structures 585 provide a surface upon which the forces can be applied to the patient 110 while the patient remains in relative comfort.

One consideration in applying the tensile forces to the occipital lobes and bones 585 is the size and shape of the patient's jaw bone and the patient's ability to open and close his/her mouth for communication during treatment. For example, certain patients may exhibit such conditions as Temporal Mandible Joint (TMJ) disorder. In such cases, the occiput structures 585 must be positioned against the occiput horns 550 such that jaw function is maintained and the patient can communicate during treatment. The occiput horns 550 must be sufficiently tall and conforming to provide resistance against the occiput structures 585 while not exceeding such height as would bring the occiput horns 550 into contact with the patient's jaw. The occiput contact surfaces 580 that contact the occiput structures 585 must additionally be both conforming and rigid, such that the contact surface 580 make firm contact against the bony structures 585 during traction or decompression treatments.

FIG. 6 illustrates a side view of the head support system of FIG. 5 used with a light therapy device 600. The light therapy device 600 includes a curved clear housing 620 carrying light sources 630. By way of example only, the light sources 630 are laser diode modules. The light therapy device 600 is positioned within the head adapter 410 underneath the convex alignment of the patient's neck 540. An air gap 625 extends between the housing 620 of the light therapy device 600 and the patient's neck 540. Any number of laser diode or light emitting elements 630 might be contained within the housing 630. The light sources 630 are arrayed across the housing 620, and the housing 620 may include various optical shapes such as lenses to collimate or diverge irradiation 640 from the light sources 630 depending on the precise configuration of the light sources 630 and intended target zone of the neck 540. Alternatively, the housing 620 need not be clear. In operation, the light therapy device 600 passes photonic energy 640 from the lighting elements 630 through slits or an opening in the head adapter 410 to the neck 540 and extended cervical spinal elements 435 of the patient 110. The light therapy device 600 may be configured to be permanently assembled within the head adapter 410 or to be removed from a compartment within the head adapter 410. Alternatively, the light therapy device 600 may be configured to be used separate from the head adapter 410. By way of example only, the head adapter 410 may be configured to be positioned on top of the light therapy device 600 and have an opening along the bottom thereof to allow photonic energy to pass from the light therapy device 600 to the neck 540.

The light therapy device 600 includes a flexible wire harness connection 635 that is connected to an outside controlling device that may provide power and data to the light therapy device 600. For example, the controlling device may communicate light intensity, frequency, on/off time, and thermal information to the light therapy device 600. Alternatively, the light therapy device 600 may include sufficient power sources and controlling mechanisms to operate independently and/or may be controlled by a wireless control device. Additionally, the wire harness connection 635 may be a flex circuit, wire bundles, or possibly fiber optic and light source couplings which deliver optical energies to the neck 540.

Returning to FIG. 4, in operation, traction or decompression treatment on the cervical spine 435 of the patient 110 begins by positioning the patient 110 correctly onto the bed or table 100 with the patient's 405 head in the head adapter 410. The body weight of the patient 110 is sufficient to anchor the body of the patient 110 on the table 100 while the patient's head 405 is allowed to be moved and extended within the head adapter 410. The head adapter 410 is configured to receive and retain the light therapy device 600 (FIG. 6) underneath the patient's neck at the cervical spine 435 such that the device 600 delivers therapeutic irradiation to the neck along the extended cervical spinal segments 435. Once the patient 110 is positioned on the table 100 and in the head adapter 410, the head support 520 (FIG. 5), table slide elements 510 (FIG. 5), and head support platform 500 (FIG. 5) can be rotated about the hinge 504 (FIG. 5) for purposes of arranging the patient 110 in a comfortable position and positioning the patient's neck in the preferred position for treatment. The operator of the traction or decompression system 400 may use the patient interface system to select the proper treatment parameters from the computing device 490 for the traction or decompression treatment. The operator may also enter data or treatment profiles for light therapy through the patient interface system to the computing device 490 in order to activate and control the light therapy controller 494.

When the operator instructs the computing device 490 to execute the user's selected treatment profiles and/or light therapy schedules, the servo-amplifier 492 activates the electromechanical actuator 470 to apply tension to the patient interface device 425 and thus the head adapter 410 in accordance with the selected treatment program. The head adapter 410 applies tension to the cervical spine 435 of the patient 110 through the occipital lobes as discussed above with respect to FIG. 5. The program may include low and high tension plateaus above 40 pounds, and may also include any number of traction or decompression treatments. Additionally, the computing device 490 may also activate the light therapy module 494 and communicate the light therapy parameters designated by the user to the light therapy device 600 (FIG. 6) via the light therapy wire harness 496 and connector 635. The light therapy device 600 irradiates the neck at the cervical spine 435 as discussed above with respect to FIG. 6. The light therapy module 494 may communicate metrics from the light therapy device 600 with the computing device 490 for adjustment of light therapy metrics during the traction or spinal decompression treatment. By way of the system 400, photonic energies may be delivered to the extended cervical spine 435 as the cervical spine 435 is stretched and articulated by the traction or decompression therapy, thus increasing the opportunity for improved absorption of the optical energies and improving the healing of the cervical spine 435.

In an alternative embodiment, a light therapy device may be used with any number of other different methods and systems of traction, decompression, or spinal elongation therapy, including manual spinal treatment, and may be used on different regions of the spine or other regions of the human body. The light therapy device can have any number of different sizes and/or shapes to accommodate the manner in which it is used with the regions of interests and with harnesses, the head adapter, or other alignment and restraining devices. Alternatively, the light therapy device may be located in or on the table itself or may be manipulated manually about the regions of interest during spinal elongation therapy.

The light therapy device is not limited to use only with elongation or stretching treatment for the spine and neck of a patient. Alternatively, the light therapy device may be configured for use with treatments that elongate or increase space between bones and/or tissue at other joints or regions of the human body. By way of example only, the light therapy device may be used with stretching or elongation treatments used at the elbow, wrist, knee, ankle or shoulder of a patient. The joint may be articulated in the course of therapy to increase or extend the area at the joint or regions, and the light therapy device is used to irradiated the exposed joint or region.

The system and method of the different embodiments provides several advantages over conventional traction or decompression therapy systems. By using a light therapy device in conjunction with traction or decompression therapy on a specific region of the spine, the system is able to articulate the region of the spine for increased exposure to the healing effects of light therapy. Specifically, increasing interdiscal space by way of traction or decompression therapy increases exposure of regions of a patient's spine to light therapy. Increased treatment area exposure results in increased photonic energy absorption, which in turn results in accelerated healing benefits related to the exposure. Thus, the patient benefits by having two therapies at once and also by an increased recovery time. Additionally, the head adapter allows the operator to position and adjust the patient's head and neck such that the patient is comfortable during treatment and that the patient's neck is in the best position for treatment. The head adapter also maintains the patients neck aligned in the direction of the applied tensile forces. The head adapter also applies the tensile forces to the head and neck through the occipital lobes in order to maximize the effect of the treatment and to maintain the patient's comfort during the treatment. Also, the head adapter may be used with or without the light therapy device.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for treating the spine of a patient by the application of tension to the spine, comprising:
an alignment device secured to the patient;
an actuator for producing tensile force;
a patient interface device extending from said alignment device to said actuator and configured to apply the tensile force from said actuator to the spine of the patient through said alignment device; and
a light therapy device positioned to irradiate a region of interest along the spine in conjunction with the application of the tensile force to the spine.

2. The system of claim 1, wherein the tension is applied as a constant force.

3. The system of claim 1, wherein the tension is applied in cycles of force.

4. The system of claim 1, wherein said alignment device includes a compartment for carrying said light therapy device.

5. The system of claim 1, wherein said light therapy device includes an array of at least one of light emitting diodes and laser diodes that irradiate the region of interest along the spine.

6. The system of claim 1, wherein said light therapy device includes a light source enclosed in a flexible housing, wherein said housing is configured to conform to the shape of the body of the patient proximate the spine.

7. The system of claim 1, further including a computer that controls the operation of the actuator and the light therapy device.

8. The system of claim 1, wherein said alignment device is configured to secure the head of the patient and carries said light therapy device therein such that said light therapy device irradiates the region of interest along the cervical spine of the patient.

9. The system of claim 1, wherein said light therapy device includes a wire harness with a connector extending therefrom, said connector being configured to be connected to at least one of a power source and controller.

10. The system of claim 1, wherein said light therapy device includes a top surface carrying light sources, said light sources being configured to vary the amount of optical energies emitted in different areas along said top surface such that different amounts of optical energies are applied to different regions of interest along the spine of the patient.

11. The system of claim 1, wherein said light therapy device includes a clear housing.

12. A system for treating the spine of a patient by the application of tension to the spine, comprising:
an alignment device secured to the patient;
an actuator for producing tensile force;
a patient interface device extending from said alignment device to said actuator and configured to apply the tensile force from said actuator to the spine of the patient through said alignment device; and
a light therapy device, wherein said alignment device is configured to retain said light therapy device such that said light therapy device is positioned proximate a region of interest along the spine and irradiates the region of interest.

13. The system of claim 12, wherein said alignment device includes a clear barrier between said light therapy device and the region of interest and said light therapy device irradiates the region of interest through said clear barrier.

14. The system of claim 12, wherein said alignment device includes a slit between said light therapy device and the region of interest and said light therapy device irradiates the region of interest through said slit.

15. The system of claim 12, wherein said light therapy device includes an array of at least one of light emitting diodes and laser diodes that irradiate the region of interest along the spine.

16. The system of claim 12, wherein said light therapy device includes a light source enclosed in a flexible housing, wherein said housing is configured to conform to the shape of the body of the patient proximate the spine.

17. The system of claim 12, further including a computer that controls the operation of the actuator and the light therapy device.

18. The system of claim 12, wherein said alignment device is configured to secure the head of the patient and carries said light therapy device therein such that said light therapy device irradiates the region of interest along the cervical spine of the patient.

19. The system of claim 12, wherein said light therapy device includes a wire harness with a connector extending therefrom, said connector being configured to be connected to at least one of a power source and controller.

20. A method of treating the spine of a patient, comprising
positioning a light therapy device proximate a region of the body of the patient along the spine;
applying tensile forces to the spine to increase separation between discs within the spine; and
applying photonic energy from the light therapy device to the region of the body of the patient along the spine where the separation between the discs has been increased in order to increase photonic energy absorption at the region.
